(19) United States
(12) Patent Application Publication
(10) Pub. No.: US 2021/0212745 A1
(43) Pub. Date: Jul. 15, 2021

(54) Vapor Ablation System with Simplified Control Over Vapor Delivery

(71) Applicant: Aqua Medical, Inc., Santa Ana, CA (US)

(72) Inventors: Jerome Jackson, Los Altos, CA (US); Roger A. Stern, Cupertino, CA (US); Virender K. Sharma, Paradise Valley, AZ (US); Lloyd Mencinger, Rancho Palos Verdes, CA (US); Scott McGill, San Ramon, CA (US); Benjamin Wang, San Leandro, CA (US)

(21) Appl. No.: 17/150,188

(22) Filed: Jan. 15, 2021

Related U.S. Application Data
(60) Provisional application No. 62/961,473, filed on Jan. 15, 2020.

(51) Int. Cl. A61B 18/04

(52) U.S. Cl. CPC A61B 18/04; A61B 2018/00577; A61B 2018/048

(57) ABSTRACT

Ablation systems and methods include an improved approach to generating heated vapor. The vapor ablation system preferably has a controller having a user interface that receives data indicative of a time of a treatment session, a pump in data communication with the controller, and a catheter having an electrode and is in fluid communication with the pump. The controller is configured to control the pump to provide a fluid to the lumen of the catheter, cause an electrical current to be delivered to the electrode in order to heat the fluid in the lumen and convert the fluid to a heated vapor, control a delivery of the fluid and a generation of the heated vapor based on the data indicative of the time and without modifying the flow rate of the fluid or the level of voltage and/or current of the electrical current based on data from sensors positioned in or on the catheter.

### CROSS-REFERENCE

The present application relies on U.S. Patent Provisional Application No. 62/961,473, entitled “Vapor Ablation System with Improved Control Over Vapor Quality and Delivery” and filed on Jan. 15, 2020 for priority and is hereby incorporated by reference.

### FIELD

The present specification relates to systems and methods configured to simplify the generation and delivery of vapor for ablation-based therapy. More particularly, the present specification relates to systems and methods for creating and delivering a continuous and reliable stream of ablative vapor for focused and consistent tissue ablation using time as a singular data input driving all subsequent operational variables.

### BACKGROUND

Ablation, as it pertains to the present specification, relates to the removal or destruction or modulation, (e.g. shrinking, tightening, remodeling, denaturing etc.), of a body tissue, via the introduction of a destructive agent, such as radiofrequency energy, laser energy, ultrasonic energy, cyroagents, or vapor, such as steam or vaporized saline. Ablation is commonly used to eliminate diseased or unwanted tissues, such as, but not limited to cysts, polyps, tumors, hemorrhoids, and other similar lesions.

Steam-based ablation systems, such as the ones disclosed in U.S. Pat. Nos. 9,615,875, 9,433,457, 9,376,497, 9,561,068, 9,561,067, and 9,561,066, which are incorporated herein by reference, disclose ablation systems that controllably deliver steam through one or more lumens toward a tissue target. One problem that all such steam-based ablation systems have is the complex interaction of multiple variables which, if not properly managed, result in the inconsistent or unreliable delivery of vapor through catheter ports, and/or the potential overheating or burning of healthy tissue.

Furthermore, the effective use of steam often requires controllably exposing a volume of tissue to steam. However, prior art approaches to steam ablation either fail to sufficiently enclose a volume being treated, thereby insufficiently exposing the tissue, or excessively enclose a volume being treated, thereby dangerously increasing pressure and/or temperature within the patient's organ. Pressure sensors located on the catheter may help regulate energy delivery, but they are not necessarily reliable and represent a critical point of potential failure in the system.

Additionally, prior art systems are often excessively costly or complex because they require the use of one or more sensors in a disposable catheter to monitor a vapor quality, a temperature or a pressure of a body tissue or area being ablated. The inclusion of such sensors increases the cost and complexity of the ablation system and requires a user to monitor for any changes in temperature and/or pressure.

It is therefore desirable to have steam-based ablation devices that integrate into the device itself safety and/or vapor control mechanisms that are simple, result in the reliable delivery of steam, and prevent unwanted burning during use. It is further desirable to be able to provide a way to better control the amount of steam to which a target tissue is exposed without relying on sensors positioned within a catheter. It is also desirable to be able to provide an automated control of steam quality without requiring feedback from sensors. Such a system could avoid the use of sensors such as those for sensing pressure, temperature, vapor quality, moisture, or any other parameter for ensuring appropriate delivery of heat.

In case conventional steam-based ablation systems encounter a technical failure and stop operating, the vapor stored in the catheter is likely to burn the patient. Therefore, it is also desirable to have a heat delivery system that delivers the heat in a manner that avoids storage of a large amount of heat that could burn the patient.

Finally, current ablation systems have inflexible port structures that make it difficult to deliver vapor directly from a catheter to tissue that is positioned largely parallel to or skew to the port in a manner that avoids losing a substantial amount of vapor. Further, it is difficult to ablate using circular footprints, as there may be gaps of untreated areas left after ablating at adjacent locations, or there may be overlap in ablation, both situations being inefficient and possibly dangerous. Therefore, there is also a need to enable focused and efficient ablation that may be easy to stack and does not miss any of the target spaces. It is also desirable to provide steam-based ablation systems and methods used to treat various conditions including pre-cancerous or cancerous tissue in the esophagus, duodenum, bile duct, pancreas, or other tissues within the gastrointestinal system.

### SUMMARY

The present specification discloses a vapor ablation system comprising: a controller having a user input configured to receive data indicative of a time of a treatment session; a pump in data communication with the controller; and a catheter in fluid communication with the pump and having an elongate shaft, a proximal end, and a distal end, the catheter comprising: at least one lumen; and at least one electrode within the lumen, wherein the controller is configured to control the pump to provide a fluid to the lumen of the catheter, wherein the controller is configured cause an electrical current to be delivered to at least one electrode in order to heat the fluid in the lumen and convert the fluid to a heated vapor, and wherein the controller is configured to control a delivery of the fluid and a generation of the heated vapor by controlling a flow rate of the fluid and a level of power, voltage and/or current based solely on the data indicative of the time of the treatment session.

Optionally, the controller is further configured to control the delivery of the fluid and the generation of the heated vapor by controlling the flow rate of the fluid and the level of power, voltage and/or current without modifying the flow rate of the fluid or the level of voltage and/or current based on data from sensors positioned in or on the catheter.

Optionally, the vapor ablation system further comprises a cap in fluid communication with the distal end of the catheter and configured to direct ablative agent from the at least one lumen to a body tissue, wherein the cap is defined by a housing enclosing a volume and wherein a sole opening in the housing is positioned on a side of the cap that is parallel to a longitudinal axis of the catheter or that is angled relative to the longitudinal axis of the catheter by 5 degrees or greater. Optionally, the cap comprises rounded or curved exterior edges or surfaces and is removably attachable to the distal end of the catheter. Optionally, the sole opening has a footprint that is polygonal in shape. Optionally, the polygonal shape comprises one of a square, a rectangle, a pentagon, or a hexagon. Optionally, the side of the cap is angled relative to the longitudinal axis of the catheter in a range of 5 degrees to 45 degrees.

Optionally, the controller is further configured to detect an actual start of heated vapor generation, as independent and separate from an initiation of fluid flow to the at least one electrode, by monitoring a change in output power, output voltage, or output current.

Optionally, the controller is further configured to automatically apply a predefined on/off duty cycle for the time of a treatment session.

Optionally, the fluid is saline.

Optionally, the controller is configured to deliver a power to the at least one electrode is in a range of 5 watts to 300 watts.

Optionally, the controller is configured to deliver a flow rate of fluid into the lumen of 2 ml per minute.

Optionally, the at least one electrode comprises a bipolar electrode.

Optionally, the controller is configured to automatically apply a fixed power/flow rate relationship during the treatment session that is not changeable based on sensed data indicative of a vapor quality, temperature, moisture level, or pressure of the heated vapor.

Optionally, the catheter does not comprise sensors configured to sense vapor quality, temperature, moisture level, or pressure of the heated vapor.

Optionally, the catheter comprises a programmable element and the controller is configured to program the programmable element based on at least one of a treatment type, power level, voltage level, current level, fluid flow rate or the treatment time. Optionally, the programmable element is a resistor.

The present specification also discloses a vapor ablation system comprising: a controller having a user interface configured to receive data indicative of a time of a treatment session; a syringe pump in data communication with the controller; a catheter in fluid communication with the syringe pump and having an elongate shaft, a proximal end, and a distal end, the catheter comprising: at least one lumen; at least one electrode within the lumen, wherein the controller is configured to control the pump to provide a fluid to the lumen of the catheter, wherein the controller is configured cause an electrical current to be delivered to at least one electrode in order to heat the fluid in the lumen and convert the fluid to a heated vapor, wherein the controller is configured to control a delivery of the fluid and a generation of the heated vapor by controlling a flow rate of the fluid and a level of power, voltage and/or current of the electrical current based on the data indicative of the time, and wherein the controller is further configured to control the delivery of the fluid and the generation of the heated vapor without modifying the flow rate of the fluid or the level of voltage and/or current of the electrical current based on data from sensors positioned in or on the catheter; and a cap in fluid communication with the distal end of the catheter and configured to direct ablative agent from the at least one lumen to a body tissue, wherein the cap is defined by a housing enclosing a volume and wherein a sole opening in the housing is positioned on a side of the cap that is parallel to a longitudinal axis of the catheter or that is angled relative to the longitudinal axis of the catheter by 5 degrees or greater.

Optionally, the controller is configured to use the data to determine a duration for which the at least one electrode receive electrical current to heat the fluid in the lumen and convert the fluid to heated vapor.

Optionally, the vapor ablation system is adapted to operate at a fixed rate of flow of fluid from the pump to the lumen of the catheter.

Optionally, the controller is configured to maintain a power delivered to the at least one electrode at a steady state by maintaining a voltage level and an impedance at a steady state.

Optionally, the controller is configured to maintain the impedance at a steady state by maintaining the rate of flow of the fluid and the salinity at a steady state.

The present specification also discloses a vapor ablation system comprising: a controller; a pump in data communication with the controller; a catheter in fluid communication with the pump and electrical communication with the controller; and having an elongate shaft, a proximal end, and a distal end, the catheter comprising: at least one lumen; at least one electrode within the lumen, wherein the pump is configured to provide a fluid to the lumen of the catheter, wherein the at least one electrode is configured to receive an electrical current from the controller to heat the fluid in the lumen and convert the fluid to a heated vapor, and wherein the controller is configured to control a quality of the heated vapor by controlling a level of voltage and/or current of the electrical current based on a time input and without relying on any temperature, pressure, moisture, or vapor quality sensors positioned on or within the catheter that is inserted into the patient's body; and, optionally, a cap in fluid communication with the distal end of the catheter and configured to direct ablative agent from the at least one lumen to a body tissue.

Optionally, the fluid is a physiologically compatible fluid containing free ions, such as including but not limited to NaCl and Ca.

Optionally, the fluid is a sodium chloride and water solution, such as saline.

Optionally, the fluid is a physiologically normal saline.

Optionally, the controller is configured to deliver a power to the at least one electrode in a range of 1 watt to 300 watts.

Optionally, the controller is configured to deliver a flow rate of fluid into the lumen of the catheter in a range of 0.1-25 ml per minute.

Optionally, the at least one electrode comprises a bipolar electrode.

Optionally, the fluid is saline and the saline has a concentration of sodium chloride ranging from 0.01% to 10%.

Optionally, the cap comprises rounded or curved exterior edges or surfaces.

Optionally, the cap comprises an outlet footprint that is polygonal in shape. The polygonal shape may comprise one of a square, a rectangle, a pentagon, a hexagon, or other geometric shape.

The aforementioned and other embodiments of the present invention shall be described in greater depth in the drawings and detailed description provided below.

### BRIEF DESCRIPTION OF THE DRAWINGS

These and other features and advantages of the present invention will be further appreciated, as they become better understood by reference to the detailed description when considered in connection with the accompanying drawings, wherein:

FIG. 1A illustrates an ablation system, in accordance with embodiments of the present specification;

FIG. 1B illustrates an ablation catheter in accordance with embodiments of the present specification;

FIG. 2A illustrates an ablation catheter for circumferential ablation in accordance with some embodiments of the present specification;

FIG. 2B illustrates a graph showing an inconsistent increase in temperature measured at vapor delivery ports of a circumferential ablation catheter;

FIG. 2C illustrates a graph showing a consistent increase in temperature measured at vapor delivery ports of a circumferential ablation catheter;

FIG. 3A illustrates an ablation catheter including a distal cap or focused ablation in accordance with embodiments of the present specification;

FIG. 3B illustrates an ablation catheter including a distal cap for focused ablation in accordance with other embodiments of the present specification;

FIG. 3C illustrates a cross-sectional side view distal cap attached to a distal end of an ablation catheter, in accordance with an embodiment of the present specification;

FIG. 3D illustrates a front-on view of a polygonal outlet of a distal cap, in accordance with an embodiment of the present specification;

FIG. 3E illustrates a front-on view of a polygonal outlet of a distal cap, in accordance with another embodiment of the present specification;

FIG. 3F illustrates a front-on view of a polygonal outlet of a distal cap attached to a distal end of an ablation catheter, in accordance with an embodiment of the present specification;

FIG. 3G illustrates a side view of a polygonal outlet of a distal cap attached to a distal end of an ablation catheter, in accordance with an embodiment of the present specification;

FIG. 4 is a flowchart listing the steps of a method of using an ablation system having a distal cap on an ablation catheter, in accordance with some embodiments of the present specification;

FIG. 5 is a flow chart illustrating an exemplary process of controlling generation of vapor in ablation device, in accordance with some embodiments of the present specification;

FIG. 6 shows an exemplary controller interface;

FIG. 7A shows an exemplary twisted pair or braided electrode, in accordance with some embodiments of the present specification; and

FIG. 7B shows an exemplary twisted multi-wire or braided electrode, in accordance with some embodiments of the present specification.

### DETAILED DESCRIPTION

Embodiments of the present specification provide systems and methods of ablation therapy for treating a variety of conditions. The embodiments of the present specification describe ablation systems and methods that achieve a high degree of safety without having sensors embedded in the catheter for monitoring parameters such as temperature, moisture, pressure, and vapor quality and a minimized chance of burning or injuring a patient.

The embodiments of the present invention are intended to be deployed in known ablation systems. An exemplary known ablation system comprises a controller, having a pump (for example, a syringe pump) attached thereto, and a catheter, comprising an elongate shaft having a proximal end, a distal end, and at least one lumen within, attached via tubing to the controller and in fluid communication with the pump. The catheter and/or tubing are disposable and together form a disposable set. The catheter includes at least one electrode, positioned within a lumen of the catheter, to provide an energy source and convert a fluid (such as saline) into a vapor (such as steam) within the lumen. The at least one electrode is positioned at or proximate the distal end or tip of the catheter. The electrodes are positioned close to an output port on the catheter such that any vapor (steam) generated travels only a short distance (for example, a few centimeters) before exiting the catheter. The catheter is also in electrical communication with the controller for supply of power to the catheter in the form of an electrical current to the at least one electrode. The catheter includes a first electrical connection port, the controller includes a second electrical connection port, and at least one conductive wire connects the first electrical connection port to the second electrical connection port.

“Treat,” “treatment,” and variations thereof refer to any reduction in the extent, frequency, or severity of one or more symptoms or signs associated with a condition.

“Duration” and variations thereof refer to the time course of a prescribed treatment, from initiation to conclusion, whether the treatment is concluded because the condition is resolved or the treatment is suspended for any reason. Over the duration of treatment, a plurality of treatment periods may be prescribed during which one or more prescribed stimuli are administered to the subject.

“Period” refers to the time over which a “dose” of stimulation is administered to a subject as part of the prescribed treatment plan.

The term “and/or” means one or all of the listed elements or a combination of any two or more of the listed elements.

In the description and claims of the application, each of the words “comprise” “include” and “have”, and forms thereof, are not necessarily limited to members in a list with which the words may be associated. The terms “comprises” and variations thereof do not have a limiting meaning where these terms appear in the description and claims.

Unless otherwise specified, “a,” “an,” “the,” “one or more,” and “at least one” are used interchangeably and mean one or more than one.

The term “controller” refers to an integrated hardware and software system defined by a plurality of processing elements, such as integrated circuits, microcontrollers, microprocessors, application specific integrated circuits, and/or field programmable gate arrays, in data communication with memory elements, such as random access memory or read only memory where one or more processing elements are configured to execute programmatic instructions stored in one or more memory elements.

The term “vapor generation system” refers to any or all of the approaches to generating steam from water described in this application.

The terms “steam”, “water vapor”, “fluid vapor” and “vapor” are used interchangeably, and refer to the gaseous phase of a fluid that is used for ablation in accordance with the various embodiments of the present specification.

The term “steam quality” or “vapor quality” refers to a ratio of steam mass to liquid mass expressed as a percentage of total mass, of the vapor.

The term “flow rate” or “volumetric flow rate” is used interchangeably, and refers to the volume of fluid that passes through the catheter embodiments of the present specification.

For any method disclosed herein that includes discrete steps, the steps may be conducted in any feasible order. And, as appropriate, any combination of two or more steps may be conducted simultaneously.

Also herein, the recitations of numerical ranges by endpoints include all numbers subsumed within that range (e.g., 1 to 5 includes 1, 1.5, 2, 2.75, 3, 3.80, 4, 5, etc.). Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present specification. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the specification are approximations, the numerical values set forth in the specific examples are reported as precisely as possible. All numerical values, however, inherently contain a range necessarily resulting from the standard deviation found in their respective testing measurements.

It should be appreciated that the devices and embodiments described herein are implemented in concert with a controller that comprises a microprocessor executing control instructions. The controller can be in the form of any computing device, including desktop, laptop, and mobile device, custom console and can communicate control signals to the ablation devices in wired or wireless form.

The present invention is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

It should be noted herein that any feature or component described in association with a specific embodiment may be used and implemented with any other embodiment unless clearly indicated otherwise.

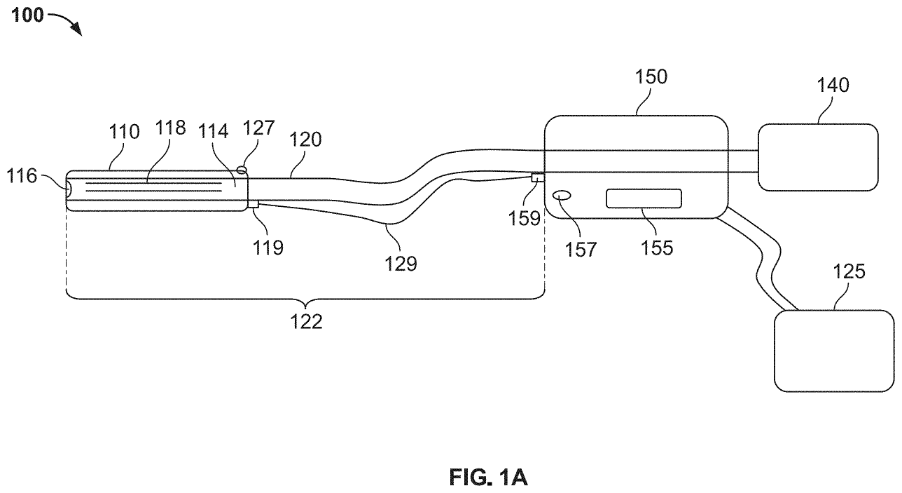

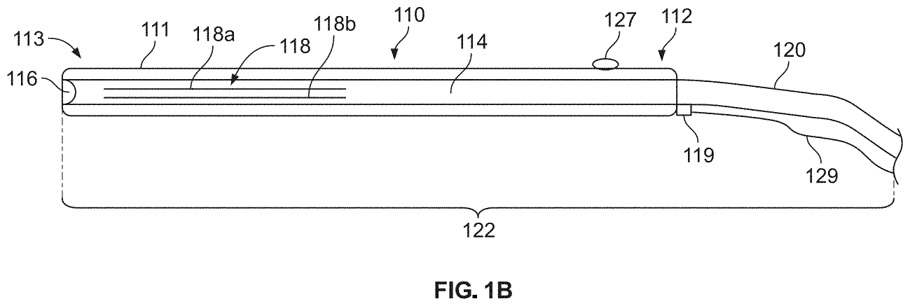

FIG. 1A illustrates an ablation system 100, in accordance with embodiments of the present specification. FIG. 1B illustrates a catheter 110 for use with the ablation system 100 of FIG. 1A. Referring to FIGS. 1A and 1B simultaneously, the ablation system 100 comprises a controller 150, a pump 140 attached to the controller 150, and a catheter 110, comprising an elongate shaft 111 having a proximal end 112, a distal end 113, and at least one lumen 114 within, attached via tubing 120 to the controller 150 and in fluid communication with the pump 140. The controller 150 includes a microprocessor 155 for controlling a rate of flow of ablative agent. In embodiments, the system 100 comprises an input device 125 in data communication with the controller 150 configured to allow a user to adjust a treatment duration. In some embodiments, the input device comprises a foot pedal and/or a graphical user interface (GUI). In some embodiments, a switch 127 on the catheter 110, or a switch 157 on the controller 150, is provided and configured to allow a user to control the flow of ablative agent.

In one embodiment, the GUI is configured to allow a user to define device, organ, and condition which in turn creates default settings for key variables, such as temperature, cycling, volume, volumetric flow rate, power, time and standard energy or radiofrequency (RF) settings, for example—RF voltage, RF current, RF power, RF impedance. In one embodiment, these defaults can be further modified by the user. The user interface also includes standard displays of all key variables, along with warnings if values exceed or go below certain levels. In embodiments, the system 100 also includes safety mechanisms to prevent users from being burned while manipulating the catheter, including markings on the catheter shaft indicating the hot or steam generation zone, insulation, and optionally, cool air flush, cool water flush, and alarms/tones to indicate start and stop of treatment.

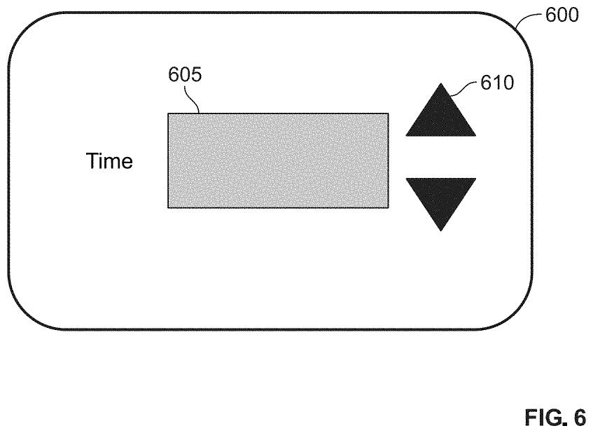

Referring to FIG. 6, in one embodiment, the controller comprises a graphical user interface 600 that just displays an input 610 configured to receive a time 605 for a chosen treatment session, which is based on the tissue being ablated and/or the therapy being provided. In one embodiment, the inputting of time, which may be determined based on the tissue being ablated and/or the therapy being provided, automatically and consequentially determines all further operational variables, including a flow rate, a voltage level, and/or a power level, as further described below.

In some embodiments, the pump 140 is a syringe pump. In various embodiments, heated vapor, generated by heating, within the catheter, an electrically conductive solution such as saline provided by the pump 140, is used as an ablative agent. Saline is preferred as a fluid for generating heated vapor, rather than water, as saline is a conductive fluid while water is not. A saline solution with a specific conductivity and resistivity is desired, as the solution requires resistivity so as to become hot and vaporize, and too high of a conductivity will prevent the heating. Additionally, surface area of the electrode is defined to optimize the amount of steam generated in a specific amount of time. Greater surface area of the electrode increases the amount of power that can be delivered, resulting in a greater amount of steam being generated in a similar amount of time with an electrode of a relatively smaller surface area. In embodiments, saline with a sodium chloride concentration in a range of 0.01% to 10% is used to optimize conductivity. In one embodiment, saline with a sodium chloride concentration of 0.9% is used.

The catheter 110 includes at least one electrode 118, positioned within the lumen 114 of the catheter 110, to deliver energy for generating heat and convert a fluid into a vapor within the lumen 114. The energy in the form of electric current (i) delivered by at least one electrode 118 heats the fluid by resistive (R) heating, which converts the fluid to vapor. In embodiments, the heat generated may be represented by the following equation:

i²*R

where i=electric current (in Amps) and R=fluid resistance (in Ohms).

The at least one electrode 118 is positioned at or proximate the distal end 113 or distal tip of the catheter 110. The at least one electrode 118 is positioned close to at least one output port 116 on the catheter 110 such that any vapor generated travels only a short distance before exiting the catheter 110. The catheter 110 is also in electrical communication with the controller 150 for supply of power to the catheter 110 in the form of an electrical current to the at least one electrode 118. In embodiments, the catheter 110 includes a first electrical connection port 119, the controller 150 includes a second electrical connection port 159, and a metal wire 129 connects the first electrical connection port 119 to the second electrical connection port 159. In some embodiments, the at least one electrode 118 comprises at least one pair of electrodes 118a, 118b or comprises at least one elongated bipolar electrode. In some embodiments, there are multiple independently controlled channels of bi-polar pairs of electrodes. In embodiments, the electrodes of the least one pair of electrodes 118a, 118b are cylindrical in shape and spaced apart from one another. Electrical current provided to the at least one electrode 118 results in a generation of heat to heat a fluid, such as saline, flowing in a lumen 114 and to convert the fluid into heated vapor. In some embodiments, multiple electrodes are positioned in “series” along the length of the catheter lumen 114. In some embodiments, multiple electrodes are positioned concentrically. In one embodiment, and as shown in FIGS. 1A and 1B, the electrode 118 has conductors on the top and bottoms sides, and fluid passes over both sides of the flat electrode assembly.

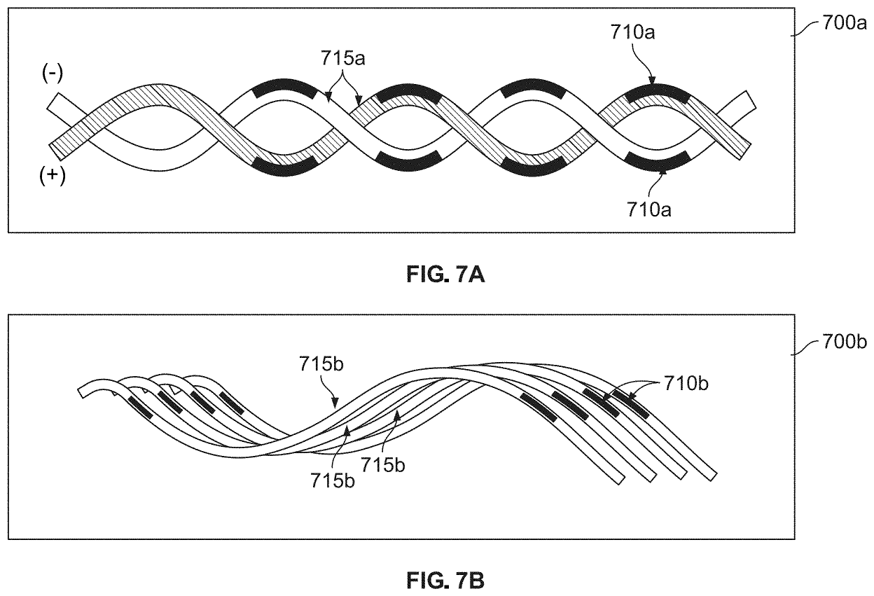

In one embodiment, referring to FIGS. 7A and 7B, the electrode 700a, 700b respectively, comprises a single twisted pair or a braid (comprising two helically twisted or interwoven wire segments 715a) or multi-twisted wires or multi-braid (comprising more than two helically twisted or interwoven wire segments 715b, preferably 3, 4, 5, 6 or more interwoven wire segments). Each wire segment in the twisted pairs or braids 715a, 715b comprises a conductive material that is covered by an insulating material, yielding twisted pairs or braids 715a, 715b having helically interwoven insulated individual wire segments. In selected locations along the length of the individual wire segments, the insulation is removed, exposing the underlying conductive material 710a, 710b. The exposed portions 710a, 710b of the individual wire segments of the twisted pairs or braids 715a, 715b are aligned such that, when evaluated along the longitudinal length of the electrode, the exposed portions of each individual wire segment may not overlap or may overlap in a range of 1% to 100%, or any increment therein.

In some embodiments, electrode configurations are defined by their respective surface areas and/or peripheral edges. In some embodiments, a vaporizing electric field is created by using a range of 1 to 25 bipolar pairs of electrodes, thereby providing a range of 4 to 100 edges, wherein four edges comprise a bipolar pair. In some embodiments, power to the at least one electrode is supplied at a range of 2 watts to 300 watts. In some embodiments, saline flow is supplied to the catheter at a range of 0.1 to 10 ml/min. In one embodiment, the ablation system operates at a 2 ml/min saline flow rate and a 50 watt power level.

The ablation systems of the present specification operate using a power/flow rate relationship to control vapor quantity and/or steam quality without further relying on sensed data from the catheter, particularly temperature, pressure, moisture, steam quality or vapor quality data. More preferably, the power/flow rate and time is automatically triggered or set based on the desired ablative effects, as previously described above. Specifically, by having the at least one electrode 118 positioned proximate the at least one output port 116 on the catheter 110, steam quantity is tightly controlled as the steam is outputted almost immediately after being produced and steam quality can be controlled. Additionally, as a result of the electrode 118 positioned proximate the output port 116, a shorter segment of the catheter, proximate the port 116 is heated. Moreover, less heat loss results in better steam quality. A high steam quality is defined as a steam wherein the amount of vapor is high relative to the amount of condensed water. In one embodiment, the heated vapor has a quality level of at least 0.10, preferably higher than 0.50, measured as the proportion of saturated vapor in a saturated condensate mixture. Steam quality can be maintained at a high level by controlling the amount of power, in the form of electrical current, being supplied to the at least one electrode 118, and by controlling a flow rate of fluid from the pump 140 to the catheter 110. As uniquely determined by the inventors, following a specific relationship between flow rate and power supplied ensures the generation of high-quality steam without requiring further control or modification by data inputted or received from one or more sensors positioned on or within the catheter. This avoids the need for any sensors positioned within, embedded within, or positioned along the length of the catheter, such as temperature, pressure, moisture, or fluid flow rate sensors, to monitor the steam quality and ensure the quality is sufficient for ablation and not out of range, possibly causing injury or under treatment. Steam quality is controlled by fine tuning two variables: a range of voltage or electrical current supplied to the at least one electrode and a range of saline flow rates. For example a voltage setting of 35 volts and a flow rate of 2.2 ml/min may result in a vapor quality on the order of 40%-60%. In a most preferred embodiment, the range of voltage supplied (solely based on a desired tissue effect input) is 25 Volts (V) to 35V, the range of current supplied (solely based on a desired tissue effect input) is 1 Ampere (A) to 5A, the range of power supplied (solely based on a desired tissue effect input) is 40 Watts (W) to 60 W, and the range of flow rate supplied (solely based on a desired tissue effect input) is 1.8 milliliter per minute (ml/min) to 2.5 ml/min. In a preferred embodiment, the range of voltage supplied (solely based on a desired time tissue effect input) is 10 Volts (V) to 55V, the range of current supplied (solely based on a desired tissue effect time input) is 0.5 Ampere (A) to 10 A, the range of power supplied (solely based on a desired tissue effect time input) is 20 Watts (W) to 100 W, and the range of flow rate supplied (solely based on a desired tissue effect time input) is 0.9 milliliter per minute (ml/min) to 4.0 ml/min.

The almost immediate generation and delivery of heated vapor from when it is produced, referred to as “just in time vapor generation and delivery”, ensures that there is a continuous flow of steam that is quickly delivered, resulting in very little heat stored within the catheter or controller and, accordingly, a safer system. In various embodiments, the ablation system stores heat at a value less than 500 J, preferably less than 100 J, defined by an amount of vapor or water equal to or less than a heated volume of 0.5 ml of saline, preferably less than 0.1 ml of saline. The embodiments of the present specification therefore provide a safety mechanism in case the system stops operating or fails, since there is no vapor that remains to be discarded. Therefore, the various embodiments prevents or dramatically reduces the risk of damage due to burns from stored heat.

In some embodiments, a patient is treated in a two-step process to ensure complete or near complete ablation of a target tissue. In some embodiments, a patient is first treated with a catheter having two positioning elements—a distal positioning element that is initially deployed followed by a proximal positioning element deployed thereafter, and a tube length with ports positioned between the two positioning elements, thereby enabling wide area circumferential ablation.

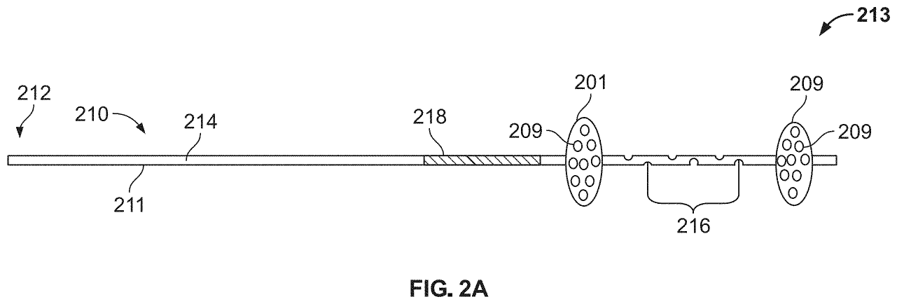

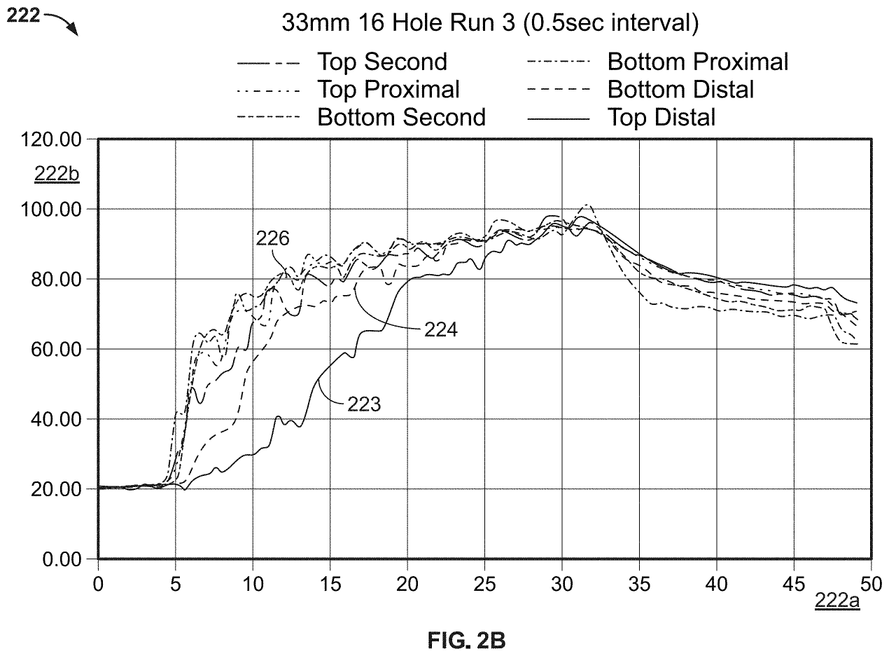

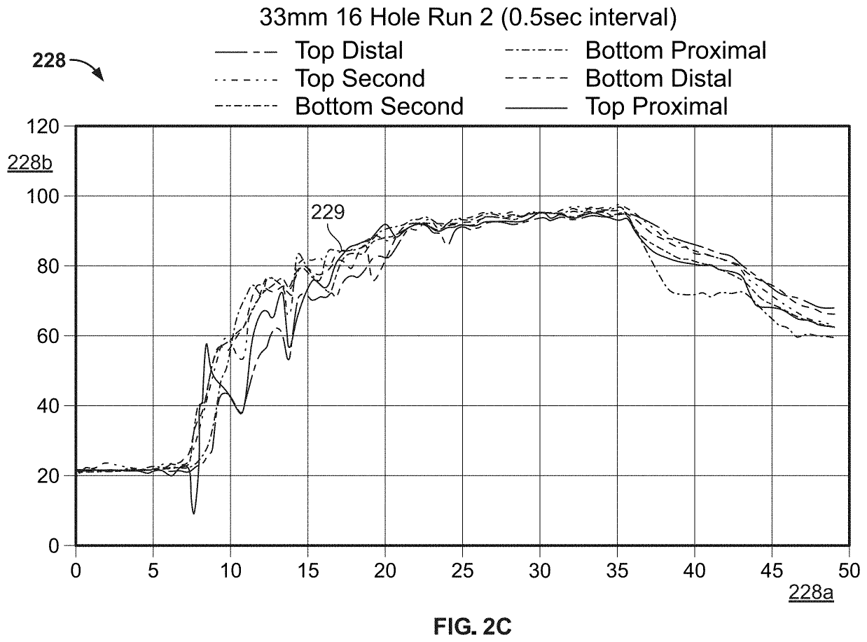

FIG. 2A illustrates an ablation catheter 200 for circumferential ablation in accordance with some embodiments of the present specification. The ablation catheter 210 includes an elongate shaft 211, a proximal end 212, a distal end 213, and at least one lumen 214. A proximal positioning element 201 is positioned proximate the distal end 213 and a distal positioning element 203 is positioned distal to the proximal positioning element 201. A plurality of ports 216 are positioned on the catheter shaft 211 in between the proximal positioning element 201 and the distal positioning element 203. At least one electrode 218 is positioned in at least one lumen 214 for converting fluid to vapor. A first contact (but preferably no seal or meaningful blocking of vapor) is created by contact of the periphery of the positioning elements 201, 203 with a patient's tissue at said distal and proximal positioning elements 201, 203. Less preferably, a seal may be created. Ablative energy, in the form of steam, is then delivered by the catheter 210 via the ports 216 into the first treatment volume, where it contacts the patient's tissue and condenses for circumferential ablation and cannot escape from the distal or proximal ends as it is blocked by the positioning elements 201, 203 (less preferably) or, preferably, escapes from the distal or proximal ends based on the configuration of the positioning elements 201, 203 or the presence of small holes or channels 209 in the positioning elements 201, 203.

The ports 216, which extend between the two positioning elements, are configured such that a surrounding chamber receives an equal distribution of vapor. In embodiments, the size, shape, direction/angle and location of the ports can vary based on position to help optimize the equal distribution of vapor. For example, the rate of temperature increase measured at various points on the internal wall of a patient's gastrointestinal (GI) tract would be substantially equal across all points. This would prevent some surfaces from receiving too much thermal energy and other surfaces from receiving too little, ensuring equal ablation.

FIG. 2B illustrates a graph 222 showing an inconsistent increase in temperature measured at vapor delivery ports of a conventional ablation catheter. An x-axis 222a illustrates the time, and a y-axis 222b illustrates the temperature (in ° C.). There are some points, depicted by curves 223, 224, that lag in terms of temperature increase relative to other points, depicted by a generally similar distribution of curves 226. In practice, these points will not get enough energy. FIG. 2C illustrates a graph 228 showing a consistent increase in temperature measured at vapor delivery ports of a circumferential ablation catheter in accordance with embodiments of the present specification. An x-axis 228a illustrates the time, and a y-axis 228b illustrates the temperature (in ° C.). The points, depicted by generally similar distribution of curves 229, shows a relatively consistent rate of increase across all points, meaning consistent energy deposition across all surfaces.

In embodiments, the circumferential ablation catheters 210 of the present specification are configured to establish an array of points defined by a specific distance from a portion of the catheter shaft 211 such that each point will experience an increase in temperature at approximately the same rate. In other embodiments, the circumferential ablation catheters 210 of the present specification are configured to establish an array of points defined by a specific distance from a portion of the catheter shaft 211 such that each point on the tissue to be ablated will experience the same temperature, from 60° C. to 90° C. to same depth, from 0.5 mm to 5 mm within five seconds of each other.

In embodiments, the ports 216 of the circumferential ablation catheters 210 of the present specification are configured such that a ratio of a surface area of port 216 openings to a surface area of catheter 210 length between the two positioning elements 201, 203 is less than 0.25, and preferably less than 0.10. The catheters 210 are configured to have a large number of holes, from 16 to 100, but not exceeding a percentage of surface area of the catheter shaft 211. In embodiments, each port 216 has a diameter ranging from 0.05 mm to 2 mm.

In some embodiments, the circumferential catheters 210 include one or more features to avoid pooling of water in the patient's organ (GI tract). “Pooling” occurs when hot water (not just steam) drips out of the ports and gathers in areas of tissue which may not be subject to ablation. As the circumferential ablation catheter is substantially horizontal when in use since the patient is lying on his or her back, hot water pools may form below the catheter and in the dependent or bottom surfaces of the GI tract. Configurations of the catheters 210 provide a check on formation of pools. In one embodiment, an outer surface of the catheter is in electrical communication with the second electrical connection port (159 in FIG. 1A) of the controller to create a heated surface. The configuration of two electrical ports may ensure that the vapor coming out of ports 216 remain in a vapor state. Additionally, in some embodiments, the ports 216 are narrow slits instead of circular ports. Narrow slits are created using laser cutting. In some embodiments, slits improve flexibility of the length between positioning elements 201 and 203. In another embodiment, the ports 216 are concentrated in certain locations where pooling is expected to occur. For example, in one embodiment, more ports 216 are positioned toward the distal end 213 of the catheter. In less preferred embodiments, steam at the distal end 213 of the catheter is pressurized by decreasing the catheter lumen 214 size or superheating the steam when it exits the catheter 210 using trumpet like nozzles at the ports 206. In still another embodiment, the ports are covered with a semi-permeable or hydrophobic material that allows gas to pass but not liquid. In some embodiments, the material is polytetrafluoroethylene (PTFE). In various embodiments, any one or combination of the above mechanisms is used to avoid all forms of pooling. In some embodiments, the saline delivery tubing and the entire fluid pathway is constructed of non-expanding (i.e. pressure rated) materials to ensure the pathway is completely void of air. The absence of air in the system or the tubing helps prevent the expansion of the system/tubing under pressure during the delivery of steam. This in turn prevents, after the delivery of steam is stopped, the drippage and pooling of fluid out of the catheter when the expanded tubing recovers.

After circumferential ablation is performed in the first step, the ablation area is examined by the physician. Upon observing the patient, the physician may identify patches of tissue requiring focused ablation. In embodiments, a circular or polygonal ablation footprint can be created, but a polygonal ablation footprint is used to make the focused ablation more efficient relative to a circular ablation footprint. A circular footprint may result in the creation of gaps or overlaps while ablating adjacent areas, which may be inefficient. In embodiments of the present specification, the polygonal ablation footprint is used, which is easy to stack and unlikely to leave gaps. After examination of the circumferential ablation area by the physician, second step is performed to provide focused ablation. During focused ablation, a second catheter with a needle or cap, hood, or disc attachment on the distal end is passed through an endoscope and used for focal ablation. In embodiments, the cap has a round or a polygonal outlet surface area. The polygonal outlet surface area may be a square, a pentagon, a hexagon, or any other type of polygon.

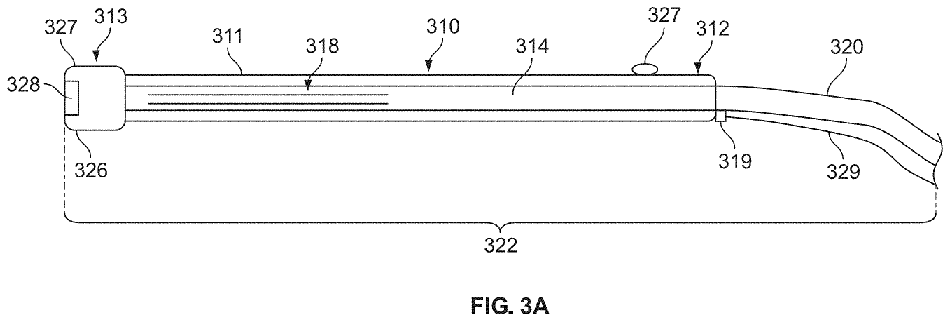

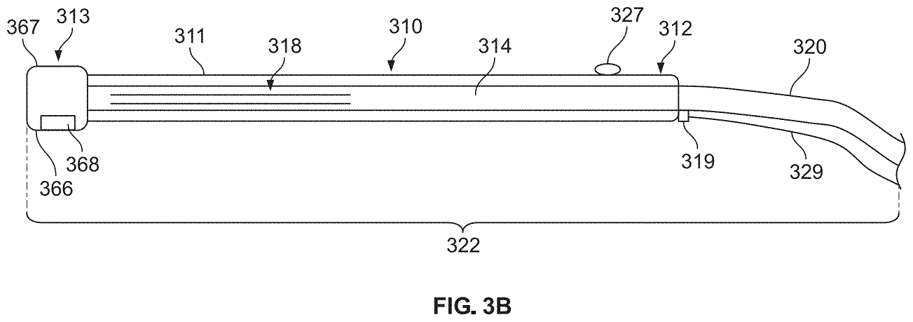

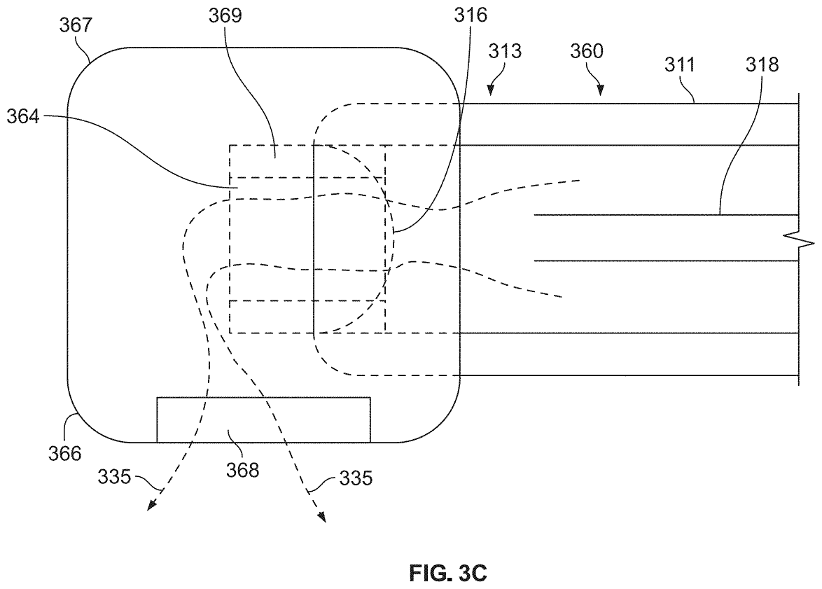

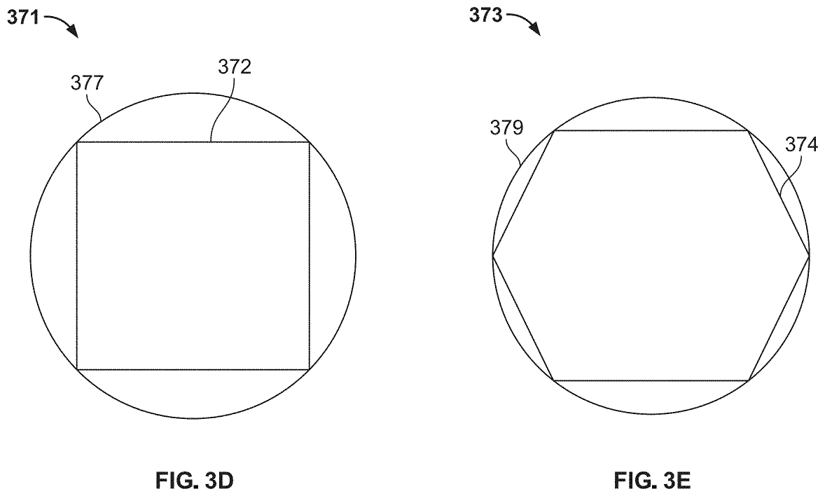

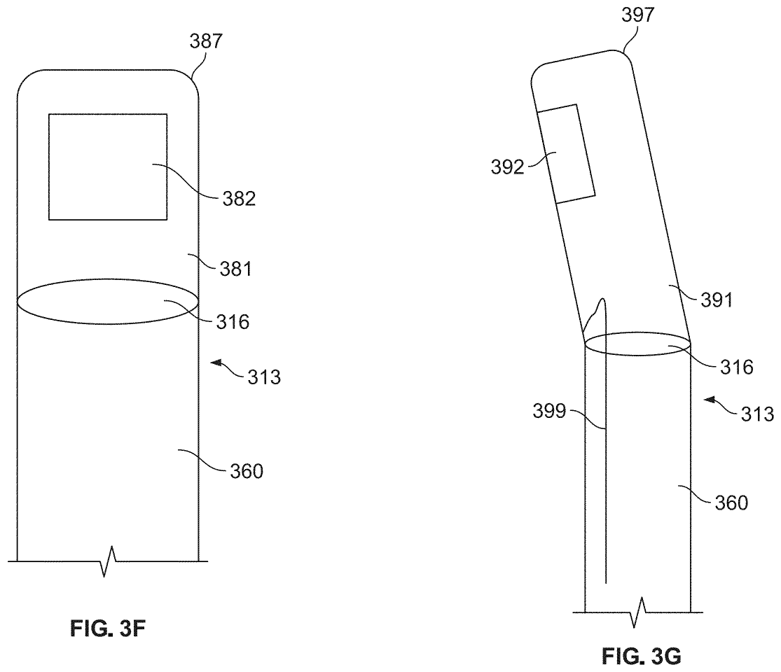

FIGS. 3A and 3B illustrate ablation catheters 310, 360 including a distal cap 326, 366 for focused ablation in accordance with embodiments of the present specification. In some embodiments, the cap 326, 366 is made of a collapsible, expanding material that can be inserted through an endoscope. In some embodiments, the cap 326, 366 can be a separate component attached to the endoscope or other surgical tools. Similar to the catheter 110 of FIG. 1B, the ablation catheters 310, 360 of FIGS. 3A and 3B include an elongate shaft 311 with a proximal end 312 and a distal end 313, at least one lumen 314 with at least one electrode 318 within, and a switch 327 for controlling vapor flow. The catheters 310, 360 are in fluid communication with a pump via tubing 320 and are in electrical communication with a controller via wire 329 connected to electrical connection port 319 at the proximal end 312. In some embodiments, the catheter 310, 360 and tubing 320 together form a disposable set 322. A distal cap 326, 366 is attached to the distal end 313 of the catheter 310, 360. The distal cap 326, 366 includes a round or a polygonal shaped outlet port 328, 368 for focused delivery of steam. In the embodiments depicted in FIGS. 3A and 3B, the outlet ports 328, 368 are rectangular or square shaped.

Referring to FIG. 3A, the outlet 328 is at a distal end of the distal cap 328. Referring to FIG. 3B, the outlet 368 is on a side of distal cap 366. It should be appreciated that the cap comprises a housing that fully encloses a volume, except for a window, which is a void or opening in the housing, positioned on a side of the cap such that it is parallel to the longitudinal axis of the catheter. An outer edge or surface 327, 367 of the distal cap 326, 366 is rounded or curved to provide an atraumatic tip and prevent injury, avoiding edges that are too sharp and could cut the patient's anatomy, for example, the gastrointestinal (GI) tract. In embodiments, the distal cap 326, 366 is enclosed except for the outlet 328, 368 that defines the ablation footprint which captures and concentrates the vapor. The footprint of outlet 328, 368 is shaped in the form of a circle or a polygon to allow for easy stacking without overlap.

FIG. 3C illustrates a cross-sectional side view of distal cap 366 attached to a distal end 313 of an ablation catheter 360 of FIG. 3B, in accordance with an embodiment of the present specification. A portion of the distal cap 366 slides over and covers a distal portion of the catheter shaft 311. The distal cap 366 includes a connector 369 with a lumen 364 that is configured to be inserted into an outlet port 316 of the catheter. The distal cap 366 includes a side outlet port 368 in a circular or a polygonal shape. Steam 335 is directed from the lumen 314 of the catheter 360, through outlet 316 and the lumen 364 of the connector 369, and out the side outlet port 368 for focused ablation. Position of the at least one electrode 318 proximate the distal end 313 of the catheter 360 ensures steam has a very short distance to travel to reach a target tissue after being generated. An outer edge or surface 367 of the distal cap 366 is rounded or curved to provide an atraumatic tip and prevent injury.

FIG. 3D illustrates a front view of a polygonal outlet 372 of a distal cap 371, in accordance with an embodiment of the present specification. The polygonal outlet 372 is square shaped and the distal cap 371 includes rounded or curved outer edges or surface 377 to provide an atraumatic tip and prevent injury. FIG. 3E illustrates a front-on view of a polygonal outlet 374 of a distal cap 373, in accordance with another embodiment of the present specification. The polygonal outlet 374 is hexagon shaped and the distal cap 373 includes rounded or curved outer edges or surface 379 to provide an atraumatic tip and prevent injury.

FIG. 3F illustrates a front view of a polygonal outlet 382 of a distal cap 381 attached to a distal end 313 of an ablation catheter 360, in accordance with an embodiment of the present specification. The polygonal outlet 382 is square shaped and the distal cap 381 includes rounded or curved outer edges or surface 387 to provide an atraumatic tip and prevent injury. Steam flows from the lumen of the catheter 360 through an outlet port 316 of the catheter 360, through the distal cap 381, and out the circular or polygonal outlet 382. FIG. 3G illustrates a side view of a polygonal outlet 392 of a distal cap 391 attached to a distal end 313 of an ablation catheter 360, in accordance with an embodiment of the present specification. The polygonal outlet 392 is rectangular shaped and the distal cap 391 includes rounded or curved outer edges or surface 397 to provide an atraumatic tip and prevent injury. Steam flows from the lumen of the catheter 360 through an outlet port 316 of the catheter 360, through the distal cap 391, and out the circular or polygonal outlet 392.

Referring to FIG. 3G, the distal cap is tilted or biased to one side, such that it is angled at least 1 degrees, preferably at least 5 degrees but less than 90 degrees, more preferably at least 10 degrees, more preferably in a range of 5 to 45 degrees, relative to the longitudinal axis of the catheter, allowing for an even more focused ablation of a target tissue. In some embodiments, the catheter 360 includes a mechanism 399 for tilting the distal cap 391 at a greater or lesser angle and for modifying the direction of the tilt. The tilted distal cap 391 with polygonal outlet 392 provides for easier positioning of the catheter 360 as the physician does not have to figure out how to bend or move the outlet surface to hit the desired target surface (given that, when first inserted, the outlet points downward, parallel to the GI tract). The physician is only required to gently push the polygonal outlet 392 against the GI tract for proper positioning. In some embodiments, the polygonal outlet 392 has a surface area in a range of 0.5 cm² to 5 cm².

The distal caps illustrated in FIGS. 3A-3G are configured to connect to the catheter distal end or tip. In some embodiments, the distal cap includes a groove and/or O-ring that attaches or snaps into the distal tip of the catheter. In some embodiments, the distal cap further comprises an additional channel that directs the vapor from the catheter lumen into the cap and toward the distal cap outlet port. In some embodiments, the catheter lumen is positioned off-center of the catheter shaft, and the distal cap further comprises a connecting member configured to insert into the catheter lumen and direct the vapor to the outlet port of the distal cap. In some embodiments, the distal cap channel has a length in a predefined range and a maximum thickness in a predefined range to fit into, and stay within, the catheter lumen.

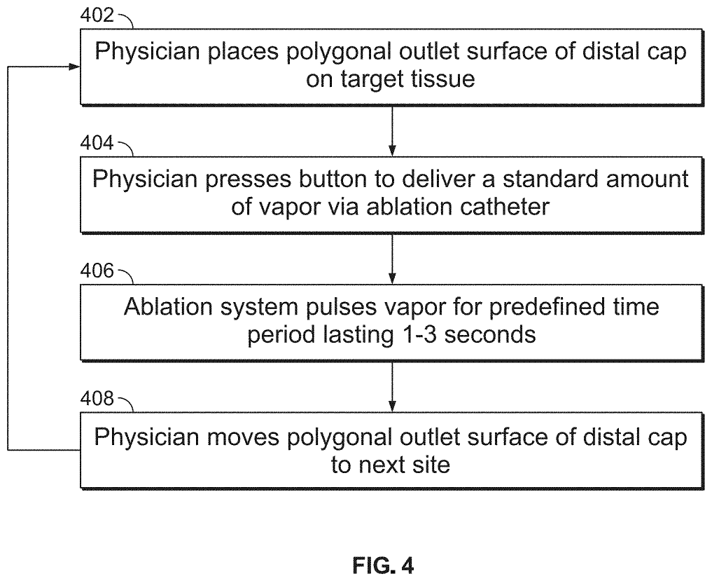

FIG. 4 is a flowchart listing the steps of a method of using an ablation system having a distal cap on an ablation catheter, in accordance with some embodiments of the present specification. At step 402, a physician places the circular or polygonal outlet surface on a target tissue, for example a portion of the patient's GI tract. At step 404, the physician presses a button (such as switch 327 of FIG. 3B or a foot pedal, such as input device 125) that causes the ablation system to pulse a standard amount of vapor. At step 406, the ablation system pulses vapor for a predefined period of time lasting in a range of 0.01-10 seconds. At step 408, the physician moves the circular or polygonal outlet surface to the next site. The physician then continues at step 402 until all focal ablation is completed. In embodiments, the ablation system is configured to output a standard amount of vapor for a predefined time period of 0.01-10 seconds as long as the physician is pressing the button (for example, foot pedal). The vapor pulse continues until the first of 1) the predefined time period (0.01-10 seconds) runs out or 2) the physician stops pressing the button (lifts foot off foot pedal).

The cap provides for directed, focal ablation and encloses the focal ablation area, optionally (but not preferentially) creating a seal and an enclosed treatment volume for ablation of the tissue. Preferably, the contact of the cap with the tissue area guides, vapor toward the treatment area, such that a portion of the patient's tissue is positioned within an area circumscribed by the attachment, but does not seal the cap over the surface of a patient's tissue, such as the esophagus or duodenum. In embodiments of the present specification, the outer surface 367 outside the circular or polygonal cap has an atraumatic shape. In one embodiment, the exterior periphery 367 of the circular or polygonal cap is rounded or curved so as to avoid sharp surfaces that could potentially damage the patient's GI tract.

In one embodiment, the flow rate of vapor out of the enclosed, or partially enclosed, volume is a predefined percentage of the flow rate of vapor into the enclosed, or partially enclosed volume from the catheter ports, where the predefined percentage is in a range of 1% to 80%, preferably less than 50%, and more preferably less than 30%. The at least one port is positioned at a distal end of the catheter such that it exits into the treatment volume when the attachment is positioned.

The devices and methods of the present specification can be used to cause controlled focal or circumferential ablation of targeted tissue to varying depth in a manner in which complete healing with re-epithelialization can occur. Additionally, the vapor could be used to treat/ablate benign and malignant tissue growths resulting in destruction, necrosis and absorption of the ablated tissue. The dose and manner of treatment can be adjusted based on the type of tissue and the depth of ablation needed. The ablation device can be used not only for the treatment of cardiac arrhythmias, Barrett's esophagus and esophageal dysplasia, flat colon polyps, gastrointestinal bleeding lesions, endometrial ablation, pulmonary ablation, but also for the treatment of any mucosal, submucosal or circumferential lesion, such as inflammatory lesions, tumors, polyps, cysts and vascular lesions. The ablation device can also be used for the treatment of focal or circumferential mucosal or submucosal lesions of any hollow organ or hollow body passage in the body. The hollow organ can be one of gastrointestinal tract, pancreaticobiliary tract, genitourinary tract, respiratory tract, heart, portions of the cardiovascular system, bladder, uterus, or a vascular structure such as blood vessels. The ablation device can be placed endoscopically, radiologically, surgically or under direct visualization. In various embodiments, wireless endoscopes or single fiber endoscopes can be incorporated as a part of the device. In another embodiment, magnetic or stereotactic navigation can be used to navigate the catheter to the desired location. Radiopaque or sonolucent material can be incorporated into the body of the catheter for radiological localization. Ferromagnetic materials can be incorporated into the catheter to help with magnetic navigation.

Ablative agents such as steam, heated gas or cryogens, such as, but not limited to, liquid nitrogen are inexpensive and readily available and are directed via the infusion port onto the tissue, held at a fixed and consistent distance, targeted for ablation. This allows for uniform distribution of the ablative agent on the targeted tissue. The flow of the ablative agent is controlled by a microprocessor according to a predetermined method based on the characteristic of the tissue to be ablated, required depth of ablation, and distance of the port from the tissue. In addition, one or more suction ports are provided to suction the ablation agent from the vicinity of the targeted tissue. The targeted segment can be treated by a continuous infusion of the ablative agent or via cycles of infusion and removal of the ablative agent as determined and controlled by the microprocessor.

The ablation systems of the present specification are configured to have reduced start-up times and priming processes. In some embodiments, impedance is measured during start-up to check whether saline is in contact with electrodes. The controller is configured to automate the impedance check and generate an error indicative of a high impedance, signifying that the saline is not in contact with the electrodes or a wire is broken. Rapid changes in impedance level at the electrodes, from high to low, are also detected to be an indication that the saline is in contact with the electrodes. Additionally, in embodiments, the controller is configured to check the power level delivered to the electrodes during the start-up. In embodiments, the controller is configured to check a radiofrequency (RF) power relative to a direct current (DC) power level. Preferably, the controller checks to determine if the RF power matches the DC power in a range of at least 50%, preferably approximately 75%. If not, the controller does not cause the treatment session to continue and indicates an error, such as an electrical short, fluid blockage, or some other error, on the graphical user interface, preferably with instructions on how to resolve the error. Preferably, the controller checks to determine if the motor current is experiencing an increase of more than 10%, or approximately 25%, indicating a stall current torque on motor. If such an increase is determined, the controller does not cause the treatment session to continue and indicates a fluid blockage on the graphical user interface, preferably with instructions on how to resolve the fluid blockage. Embodiments of the present specification also eliminate the need for a pressure sensor. Any fluid blockage or flow issues are detected by measuring resistance of fluid flow at the pump. Relatively higher current needed to push the syringe may indicate a blockage. The detection is therefore performed by the controller, eliminating the need to include a pressure sensor with the catheter.

In embodiments, the controller is configured to automatically flush the catheter before insertion into the patient, during start-up. Automatic flushing by the controller bypasses the need for the user to activate the flushing and manually stop the flushing once the water comes out of the catheter. In embodiments, start-up time is decreased by delivering a high power on the order of 2 times to 4 times the normal treatment power level (which could be in a range of 150 W to 300 W), to kick start the steam and then decreasing the power to a steady state level (which may be approximately 60 W or otherwise defined as a power level that does not vary over a time period, such as 5, 10, or more seconds, by more than 10%, and preferably by more than 5%) delivered to the electrodes. The power delivered is also automatically controlled by the controller.

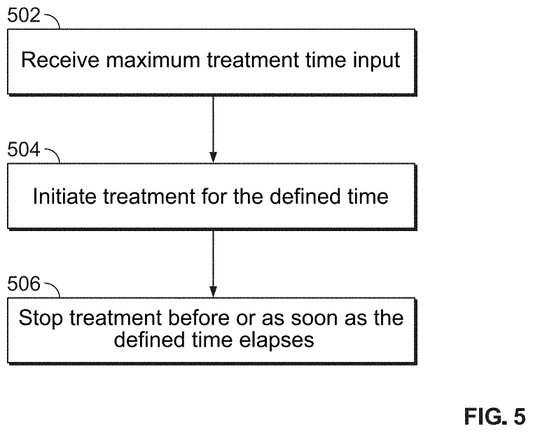

Embodiments of the ablation system of the present specification provide methods and systems for controlled generation of vapor for ablation. Referring again to FIG. 1A, in some embodiments, the ablation system 100 is responsible for generating an electrical current and for applying force to the pump 140 that provides a flow of ablative agent such as saline into the lumen 118 of the catheter 110. The electrical current is passed to electrodes 118a/118b within the lumen 118, with the use of an electrical port 127 that is directly connected to an electrical port 157 on the controller 150. FIG. 5 is a flow chart illustrating an exemplary process of controlling generation of vapor in ablation device 100, in accordance with some embodiments of the present specification. At step 502, a user interface (UI) is available in the form of an input device to the controller 150. A user, such as a clinician, interfaces with the UI to set a maximum treatment time for the vapor ablation process. In embodiments, the treatment time is set prior to initiating the treatment. The UI may provide a touch screen, buttons, or a combination of both, and a display, to enable the user to input and view the time period being set for the treatment, as shown in FIG. 6. At step 504, the treatment time input by the user is used by the controller to determine the amount of energy needed to ablate the target tissue. Treatment is initiated when the controller starts supplying power to operate the electrodes 118, which in turn generates vapor by heating the ablation fluid supplied by the pump 140. The power supplied to the ablation device 100 and the rate of flow of the ablation fluid from pump 140, are constant, therefore the amount of time set by the user, which is a function of the tissue being ablated, determines the amount of energy that is delivered at the target site during ablation. An impedance/resistance of the ablation fluid is consistent since the flow rate is stable and the salinity (conductivity) of the ablation fluid is consistent. The following equation is used to represent the total energy that is delivered:

Total energy delivered=Power×Time, where Power is a function of the current and the voltage supplied to the device 100 from a power source.

At step 506, controller 150 stops vapor generation and therefore discontinues the delivery of energy, when the set time period is completed. The vapor generation stops as the controller 150 disables the power supply to electrodes 118 in the catheter 110, when the pre-defined time elapses. Optionally, the user may stop the process before the set time period, through manual intervention. In one embodiment an input device provided in the form of an option or a button on the UI, or the foot pedal, is used to intervene and stop the ablation process. Embodiments of the present specification are able to limit the maximum dose of ablation that is delivered by automatic shutting off of the generation of vapor based on a maximum treatment dose. The maximum dose is input into the device 100 as a function of time at step 502. As an additional safety measure, the device 100 enables the user to discontinue delivery of energy at any time during the treatment, even before the maximum dose is reached, by disabling the power supply using the UI or by releasing the pedal. In some embodiments, the controller 150 is programmed to deliver therapeutic ablation treatment repeatedly for a pre-defined duration, where each treatment is for the set time period and is interleaved with a gap of another pre-defined time when the vapor delivery is stopped.

While the catheter preferably does not comprise a sensor to sense the flow rate, temperature, pressure, vapor quality or moisture level, in one embodiment, the catheter may comprise a programmable element or a component with a characteristic that can be measured, such as a resistor. Preferably, the value stored in the programmable element or measured component value, such as a resistor value, can be automatically set by the controller. In one embodiment, the controller is configured to program the value, such as a resistance value, based on a type of treatment or set treatment variables, such as power, voltage, current, fluid flow rate and/or treatment time, as discussed throughout this application.

### Vapor Generation Control Algorithms

In embodiments, the controller is programmed to automate the generation of vapor for priming and for the therapeutic treatment. In one embodiment, a control algorithm emanates from control signals and measurements associated with RF energy delivery to ablation fluid and the resulting transformation of the ablation fluid (water or saline) into the vapor or steam phase. The controller controls voltage, current and/or power to heat the ablation fluid and generate fluid vapor, and measures the results of these control signals. The measured signals are used to further control or optimize the characteristics of the vapor or steam. Accordingly, the vapor generation process encompasses a series of steps that are performed prior to using the ablation device. A control or an output voltage is set by the user, and the resultant current is measured. The controller then calculates the impedance and resulting power that is delivered to the ablation fluid. Similarly, a control or output current is set, the resulting voltage is measured, and power delivered to the ablation fluid and the resulting impedance are calculated. Further, a control or output power is set, the voltage or current levels are adjusted to achieve the desired power to be delivered to the ablation fluid, and the resulting impedance is calculated. Still further, a control or output voltage is set, the resultant current is measured, the power delivered to the fluid to produce vapor and the resulting impedance are calculated. In embodiments, the control voltage and/or the control current can be adjusted to modify the power delivered to the fluid as well as the amount or quality of steam that is generated.

As mentioned here, the controller is enabled to measure and control steam generation using one or a combination of the stated steps. In the cases where the controller a) sets the control voltage and measures the current, the impedance is calculated, and b) sets the control current and measures the voltage, the impedance is calculated, a change in impedance is calculated when vapor generation is initiated resulting in an associated change in the impedance. The change in impedance may reflect as a sharp increase in impedance, for example in the form of a ‘step change’. The amount of the change in impedance may depend on the intrinsic impedance of the fluid. For example, for saline the intrinsic impedance at the RF output frequency of 460 kHz for one steam chamber geometry is on the order of 2 ohms. When generating vapor, the impedance at 460 kHz is highly variable and on the order of, for example, 10-60 ohms or larger, depending on the vapor quality and the vapor power.

A start of vapor or steam generation may be detected based on one or more characteristics derived from measurement of impedance. In one embodiment, step change in impedance from approximately 2 ohms to a minimum average value of approximately 15 ohms may indicate vapor generation. In another embodiment, change in the characteristics of the impedance calculations from a semi constant or slowly changing value on the order of 1-3 ohms to a highly variable value ranging from approximately 10 ohms to 60 ohms, or more, indicates vapor generation. The change in impedance is caused by the conversion of fluid to vapor as the random process of boiling occurs with the resulting vapor that is in contact with the electrode surfaces being less conductive than the fluid that was in contact with the electrode surfaces. Since boiling is random, the resulting impedance changes randomly from a low value to high values and values between the low and high ranges.

In the case where the controller:

a. sets the control voltage and measures the current,

b. sets the control current and measures the voltage

the RF output power is calculated. A change in output power is detected when the vapor generation begins. The change in output power is reflected as a sharp decrease, or a ‘step change’ decrease in output power. The amount of the change may depend on the intrinsic impedance of the ablation fluid. For example, for saline the intrinsic impedance at the RF output frequency of 460 kHz is on the order of 2 ohms for a certain model of the steam chamber geometry. When delivering the vapor the impedance at 460 kHz is variable and on the order of, for example, 10-60 ohms or larger, depending on the vapor quality and the vapor power.

The controller may detect the start of actual vapor or steam generation based on one or more characteristics derived from measurement of power. In one embodiment, a decrease or step change in output power from approximately 100-200 watts to a minimum average value of approximately 40-50 Watts indicates vapor generation. In another embodiment, a change in the characteristics of the power calculations from a semi constant or minimally changing value on the order of 100-200 Watts to a variable value ranging from approximately 10 Watts to 60 Watts, is an indication of vapor generation. While the changes in the calculated power depend on the voltage setting but the form of the changes are similar. For example, if the voltage is on the order of 30 volts, the average power is calculated on the order of 50 Watts. If the voltage is on the order of 15 volts, the average power is calculated on the order of 20 Watts. Accordingly, in one embodiment, the controller is configured to detect the actual start of vapor generation, as independent and separate from the initiation of fluid flow to a heating chamber, by monitoring a change in output power, such as a decrease in a first output power from a range of 100 to 200 watts to a second output power in a range of 60 watts or less.

In the case where the controller sets the power and adjusts the current and/or the voltage to achieve the set power, a change in output voltage and/or current is detected when the vapor generation begins. The change in output voltage and/or current or resistance is reflected as a sharp increase, or a ‘step change’ increase in voltage, or sharp decrease or a ‘step change’ decrease in current. The amount of the change may depend on the intrinsic impedance of the ablation fluid. For example, for saline the intrinsic impedance at the RF output frequency of 460 kHz is on the order of 2 ohms for a certain model of the steam chamber geometry. When delivering the vapor the impedance at 460 kHz is variable and on the order of, for example, 10-60 ohms or larger, depending on the vapor quality and the vapor power. Accordingly, in one embodiment, the controller is configured to detect the actual start of vapor generation, as independent and separate from the initiation of fluid flow to a heating chamber, by monitoring a change in output voltage or current, such as an increase in output voltage or decrease in current.

The controller may detect the start of actual vapor or steam generation based on one or more characteristics derived from measurement of power. In one case, if there is a decrease or step change in output power from approximately 100-200 watts to a minimum average value of approximately 40-50 watts, change in the characteristics of the power calculations from a semi constant or minimally changing value on the order of 100-200 watts to a highly variable value ranging from approximately 10 watts to 60 watts, is an indication of vapor generation. While the changes in the calculated power depend on the voltage setting, the directionality of the changes are similar. For example, if the voltage is on the order of 30 volts, the average power is calculated on the order of 50 Watts. If the voltage is on the order of 15 volts, the average power is calculated on the order of 20 Watts.

In another case, if there is an increase or step change in resistance from approximately 2-3 ohms to a minimum average value of approximately 8-20 Ohms, change in the characteristics of the resistance values from a semi constant or minimally changing value on the order of 2-3 Ohms to a highly variable value ranging from approximately 8 Ohms to 20 Ohms is an indication of vapor generation. In yet another embodiment, if there is an increase or step change in voltage from approximately 7 volts to a minimum average value of approximately 30 volts, change in the characteristics of the output voltage from a semi constant or minimally changing value on the order of 7 volts to a highly variable value ranging from approximately 30 volts to 34 volts, is an indication of vapor generation. Accordingly, in one embodiment, the controller is configured to detect the actual start of vapor generation, as independent and separate from the initiation of fluid flow to a heating chamber, by monitoring a change in the variability of resistance, a change in the variability of current, a change in the variability of voltage, or a change in the variability of power, such as an increase in variability.

For each of the above-stated modes of controls involving voltage, current, and power, the voltage source control can be replaced with a current source control and associated measurements of voltage, to achieve the same control responses. Alternatively, for each of the control modes the current source control can be replaced with a voltage source control and associated measurements of current, to achieve the same control responses. Additionally, voltage or current control can be replaced with power control and the associated changes in the control voltage or control current can be achieved.

In embodiments of the present specification, the RF voltage, current and/or power delivery is interrupted, and therefore not constant, during the treatment time, which has the effect of decreasing the energy delivered and may alter or reduce the resultant temperature of the fluid or vapor, and/or reduce the rate of fluid or vapor temperature increase during the treatment time. The interruption in RF energy delivery may occur in a periodic or non-periodic manner to result in the desired energy delivery profile and/or rate of temperature increase. Additionally, the fluid flow rate may be modified in a periodic or a non-periodic manner to adjust the energy delivery rate and/or rate of temperature rise.

Embodiments of the present specification enable the controller to be programmed to control a pulsed delivery of ablative energy in the form of vapor, and the temperature response, during a therapeutic treatment. For this purpose, the user may set a treatment time, which would then automatically result in the setting of voltage, current, and/or power at a level that is desired for ablating a target tissue in accordance with the set time. In one embodiment, for a specific configuration of the steam chamber of an ablation device, the control voltage, current or power is automatically set for a portion of the inputted time period, where that portion is approximately 250 milliseconds (ms). The controller may then automatically stop the RF delivery for a period of approximately 250 ms. The duty cycle of 250 ms of enabling RF delivery and 250 ms of disabling RF delivery is repeated. In this way, a pulsed delivery of RF energy is provided to the ablation device that represents a periodic and symmetric 50:50 duty cycle. In another embodiment, the control voltage, current or power automatically set by the inputted time is delivered for a period of approximately 1000 ms, followed by a gap of approximately 300 ms when the RF delivery is stopped, followed again by RF delivery of 1000 ms. In this embodiment, repeating the duty cycle of 25:75 also delivers pulsed ablative energy. It should be appreciated that the duty cycle of on:off may be divided into 5:95 to 95:5 as a proportion of the inputted treatment time. As such, the controller is configured to automatically translate an inputted treatment time into a on:off duty cycle in the aforementioned ranges.

In further embodiments, the duration of enabling and disabling the RF delivery may be varied within a treatment duration. In one embodiment of a pulsed delivery cycle, the control voltage, current, or power is delivered for 100 ms, stopped for 100 ms, delivered again for 100 ms, then stopped for 300 ms. Repeating this pulsed delivery pattern represents a periodic and asymmetric duty cycle of 50:50:25:75. Therefore, in embodiments, the duty cycle may be adjusted to result in a multitude of energy delivery and/or rate of fluid and/or vapor temperature rise results.

While not preferred, the output from the ablation device is measured during the treatment to provide additional controls, in accordance with some embodiments. In some embodiments, a flow rate of the pump, such as a syringe pump, is adjusted during the treatment. Adjusting the flow rate may optimize vapor quality so as to increase average impedance. The flow rate is adjusted to increase or decrease based on the required quality of vapor. An increased flow rate increases the vapor quality, whereas a decreased flow rate decreases the vapor quality. In one scenario, the flow rate is decreased or even ceased so as to minimize fluid delivery after the ablative vapor is delivered to the target tissue, such as in a pulsed delivery of treatment. In embodiments, the controller received output signals, such as temperature and impedance during vapor generation, and uses them to adjust or control the fluid flow rate from the pump. Output measurements from the ablation device are used to monitor consistency of vapor generation, and fluid flow rate control signals can be adjusted to compensate for output variations.

In one embodiment, the fluid flow rate is adjusted by the controller to a first rate (R1) before detecting the generation of vapor; and to a second rate (R2) after detecting the generation of vapor. Such modification of the rate of flow from syringe pump can minimize delivery of fluid or low quality vapor to the treatment site. In one example, the fluid flow rate (R1) before detection of vapor production may be approximately 0.01 milliliter per minute (ml/min) to 1.0 ml/min and the fluid flow rate (R2) after detection of vapor may be approximately 2.0 ml/min to 2.2 ml/min. The flow rate may vary for different geometries of steam chambers. Multiple combinations are possible depending on the desired vapor power output, quality and amount of liquid delivered to the treatment site.

Configurations for the various catheters of the ablation systems of the embodiments of the present specification may be different based on the tissue or organ systems being treated. Distribution and depth of ablation provided by the systems and methods of the present specification are dependent on the duration of exposure to steam, the ablation size, the temperature and/or quality of the steam, the contact time with the steam, and the tissue type.

The above examples are merely illustrative of the many applications of the system of the present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A vapor ablation system comprising:
a controller having a user input configured to receive data indicative of a time of a treatment session;
a pump in data communication with the controller; and
a catheter in fluid communication with the pump and having an elongate shaft, a proximal end, and a distal end, the catheter comprising:
at least one lumen; and
at least one electrode within the lumen,
wherein the controller is configured to control the pump to provide a fluid to the lumen of the catheter, wherein the controller is configured cause an electrical current to be delivered to at least one electrode in order to heat the fluid in the lumen and convert the fluid to a heated vapor, and wherein the controller is configured to control a delivery of the fluid and a generation of the heated vapor by controlling a flow rate of the fluid and a level of power, voltage and/or current based solely on the data indicative of the time of the treatment session.

2. The vapor ablation system of claim 1, wherein the controller is further configured to control the delivery of the fluid and the generation of the heated vapor by controlling the flow rate of the fluid and the level of power, voltage and/or current without modifying the flow rate of the fluid or the level of voltage and/or current based on data from sensors positioned in or on the catheter.

3. The vapor ablation system of claim 1, further comprising a cap in fluid communication with the distal end of the catheter and configured to direct ablative agent from the at least one lumen to a body tissue, wherein the cap is defined by a housing enclosing a volume and wherein a sole opening in the housing is positioned on a side of the cap that is parallel to a longitudinal axis of the catheter or that is angled relative to the longitudinal axis of the catheter by 5 degrees or greater.

4. The vapor ablation system of claim 3, wherein the cap comprises rounded or curved exterior edges or surfaces and is removably attachable to the distal end of the catheter.

5. The vapor ablation system of claim 3, wherein the sole opening has a footprint that is polygonal in shape.

6. The vapor ablation system of claim 5, wherein the polygonal shape comprises one of a square, a rectangle, a pentagon, or a hexagon.

7. The vapor ablation system of claim 3, wherein the side of the cap is angled relative to the longitudinal axis of the catheter in a range of 5 degrees to 45 degrees.

8. The vapor ablation system of claim 1, wherein the controller is further configured to detect an actual start of heated vapor generation, as independent and separate from an initiation of fluid flow to the at least one electrode, by monitoring a change in output power, output voltage, or output current.

9. The vapor ablation system of claim 1, wherein the controller is further configured to automatically apply a predefined on/off duty cycle for the time of a treatment session.

10. The vapor ablation system of claim 1, wherein the fluid is saline.

11. The vapor ablation system of claim 1, wherein the controller is configured to deliver a power to the at least one electrode is in a range of 5 watts to 300 watts.

12. The vapor ablation system of claim 1, wherein the controller is configured to deliver a flow rate of fluid into the lumen of 2 ml per minute.

13. The vapor ablation system of claim 1, wherein the at least one electrode comprises a bipolar electrode.

14. The vapor ablation system of claim 1, wherein the controller is configured to automatically apply a fixed power/flow rate relationship during the treatment session that is not changeable based on sensed data indicative of a vapor quality, temperature, moisture level, or pressure of the heated vapor.

15. The vapor ablation system of claim 1, wherein the catheter does not comprise sensors configured to sense vapor quality, temperature, moisture level, or pressure of the heated vapor.

16. The vapor ablation system of claim 1, wherein the catheter comprises a programmable element and wherein the controller is configured to program the programmable element based on at least one of a treatment type, power level, voltage level, current level, fluid flow rate or the treatment time.

17. The vapor ablation system of claim 16, wherein the programmable element is a resistor.

18. A vapor ablation system comprising:
a controller having a user interface configured to receive data indicative of a time of a treatment session;
a syringe pump in data communication with the controller;
a catheter in fluid communication with the syringe pump and having an elongate shaft, a proximal end, and a distal end, the catheter comprising:
at least one lumen;
at least one electrode within the lumen,
wherein the controller is configured to control the pump to provide a fluid to the lumen of the catheter, wherein the controller is configured cause an electrical current to be delivered to at least one electrode in order to heat the fluid in the lumen and convert the fluid to a heated vapor, wherein the controller is configured to control a delivery of the fluid and a generation of the heated vapor by controlling a flow rate of the fluid and a level of power, voltage and/or current of the electrical current based on the data indicative of the time, and wherein the controller is further configured to control the delivery of the fluid and the generation of the heated vapor without modifying the flow rate of the fluid or the level of voltage and/or current of the electrical current based on data from sensors positioned in or on the catheter; and
a cap in fluid communication with the distal end of the catheter and configured to direct ablative agent from the at least one lumen to a body tissue, wherein the cap is defined by a housing enclosing a volume and wherein a sole opening in the housing is positioned on a side of the cap that is parallel to a longitudinal axis of the catheter or that is angled relative to the longitudinal axis of the catheter by 5 degrees or greater.

19. The vapor ablation system of claim 18, wherein the controller is configured to use the data to determine a duration for which the at least one electrode receive electrical current to heat the fluid in the lumen and convert the fluid to heated vapor.

20. The vapor ablation system of claim 18, wherein the vapor ablation system is adapted to operate at a fixed rate of flow of fluid from the pump to the lumen of the catheter.

21. The vapor ablation system of claim 18, wherein the controller is configured to maintain a power delivered to the at least one electrode at a steady state by maintaining a voltage level and an impedance at a steady state.

22. The vapor ablation system of claim 18, wherein the controller is configured to maintain the impedance at a steady state by maintaining the rate of flow of the fluid and the salinity at a steady state.
